(19) United States
(12) Patent Application Publication
(10) Pub. No.: US 2024/0091129 A1
(43) Pub. Date: Mar. 21, 2024

(54) COMPOSITION FOR TREATING SKIN TISSUE AND A METHOD MAKING THE SAME

(71) Applicants: Raymond S. James, Livingston, NJ (US); Camila Mello, Livingston, NJ (US)

(72) Inventors: Raymond S. James, Livingston, NJ (US); Camila Mello, Livingston, NJ (US)

(21) Appl. No.: 18/470,822

(22) Filed: Sep. 20, 2023

Related U.S. Application Data
(60) Provisional application No. 63/376,509, filed on Sep. 21, 2022.

(51) Int. Cl. A61K 8/92; A61K 8/9789; A61Q 19/00

(52) U.S. Cl. CPC A61K 8/927; A61K 8/922; A61K 8/9789; A61Q 19/00

(57) ABSTRACT

A method for treating skin tissue, the method including dermally administering to a human or animal a portion of a composition having about 10 g of a beeswax; about 18 g of a mango butter; about 60 g of an extract of extra virgin coconut oil; about 8 g of a shea butter; about 1 ml of lavender oil; about 1 ml of geranium oil; and about 2 ml of blue tansy oil.

### CROSS-REFERENCE TO RELATED APPLICATION

This application claims the benefit of priority of U.S. provisional application No. 63/376,509, filed Sep. 21, 2022, the contents of which are herein incorporated by reference.

### BACKGROUND OF THE INVENTION

The present invention relates to skin tissue and, more particularly, to a composition for treating skin tissue as well as a method of making the composition for treating skin tissue.

Skin is constantly damaged and injured. Skin tissue becomes red, inflamed, and dry under such conditions.

Presently available compositions and methods for treating skin and skin tissue are full of toxic components which are not beneficial to and in some cases inhibit, retard, or are otherwise counterproductive to the healing process.

As can be seen, there is a need for a non-toxic composition for treating skin and skin tissue and a method of making the same.

### SUMMARY OF THE INVENTION

A composition compatible with skin for promoting skin tissue healing, wherein the naturally derived ingredients of the composition are high in emollients, antioxidants, vitamins A and E, and oleic acid along with other plant-based ingredients. The combination of these ingredients encourages skin regeneration, creates a softening/calming effect that reduces flare-ups by nourishing the skin tissues.

The present invention embodies a 100% chemical free composition, wherein all its ingredients have zero toxins and so serve a beneficial purpose in promoting the healing process.

In one aspect of the present invention, a method for treating skin tissue includes dermally administering to a human or animal a portion of a composition having about 10 g of a beeswax; about 18 g of a mango butter; about 60 g of an extract of extra virgin coconut oil; about 8 g of a shea butter; about 0.25 g of lavender oil; about 0.25 g of geranium oil; and about 0.5 g of blue tansy oil.

In another aspect of the present invention, method for treating skin tissue, the method comprising dermally administering to a human or animal a portion of a composition comprising compositional components: beeswax; mango butter; an extract of extra virgin coconut oil; a shea butter; lavender oil; geranium oil; and blue tansy oil, wherein said component were mixed in a sequence based on a density of each compositional components, wherein the sequence is defined by most to least dense compositional components, and wherein the beeswax, mango butter, and the shea butter are melted prior to being mixed with other said compositional components.

These and other features, aspects and advantages of the present invention will become better understood with reference to the following drawings, description and claims.

### BRIEF DESCRIPTION OF THE DRAWINGS

FIG. 1 is a flowchart for an exemplary embodiment of the present invention; and

FIG. 2 is a schematic view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a composition for treating damaged and injured skin tissue, reducing redness and inflammation, and nourishing dry skin tissue. The present invention also contemplates as method of making such a composition.

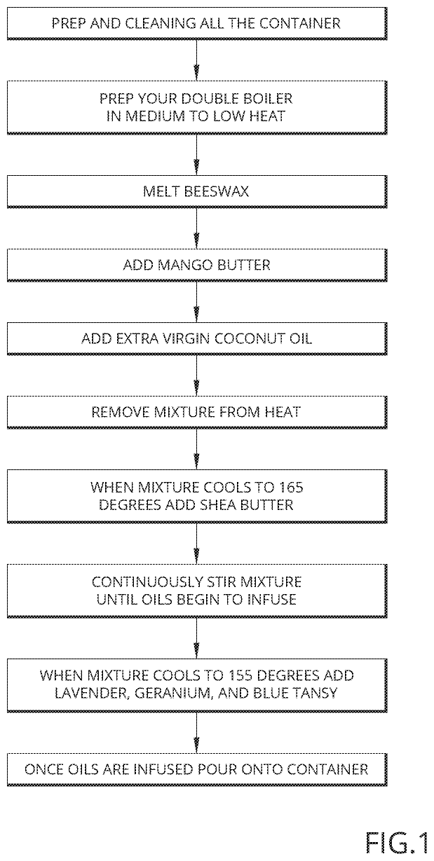

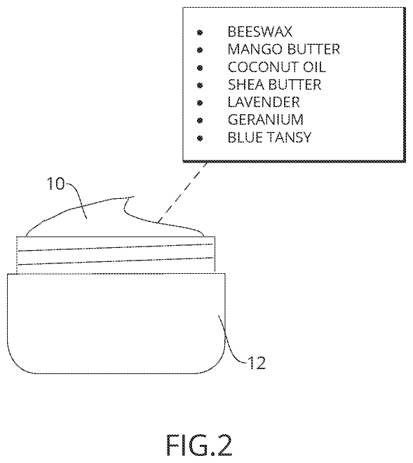

Referring to FIGS. 1 and 2, the present invention may be non-toxic and include only natural ingredients. All ingredients may serve a beneficial purpose in promoting the healing process.

The present invention may have a competitive shelf life relative to synthetic compositions of the prior art, yet the present invention is made from all natural, non-toxic ingredients. By having naturally derived ingredients the composition embodied by the present invention does not hinder the healing process of the skin tissue to which it was applied.

The present invention may be formed according to the method steps of the present invention as illustrated in FIG. 1. Bottles may be prepared and cleaned. A cleaning process lowers the number of potential contaminants that can affect the overall outcome. A pot on a heater/boiler or other heating element may be slowly progressively brought to a first temperature of approximately 80 to 160 degrees Fahrenheit to avoid burning any of the systemic components.

In one embodiment, 10-25 grams of beeswax may be placed into the boiler and melted. Beeswax may be melted first as it is densest systemic component in terms of consistency and requires complete melting at 140-150 degrees Fahrenheit. The present invention proceeds in adding system components based on their density, where the systemic components are added in sequence starting with the densest systemic component (beeswax) and ending with the least dense systemic component (blue tansy oil).

18-25 grams of mango butter may be melted in a separate pot and once melted, is mixed with the beeswax to form a first admixture. All butter-based systemic components are melted separately then added together in an ice water bath to cool down to be mixed with a hand mixer to obtain the whipped consistency. The inventor has discovered that the skin care composition 10 is easier to make this way. Additionally, or alternatively, a manufacturer can melt all butter-based systemic components in separate containers until they achieve the recommended melting points, then one can add all melted butter to the beeswax container to form the first admixture, and then add that container to ice water. The first admixture is then re-heated so that the mango butter and beeswax is brought to a combined liquid form together.

60-90 grams extra virgin coconut oil may be introduced into the liquid form of the first admixture (beeswax and mango butter combination), thereby forming a second admixture.

The pot with the above combined ingredients may be removed from heat and allowed to cool. 8-20 grams of shea butter is then introduced and combined with the second admixture at approximately 90-165 degrees Fahrenheit, thereby initiating a third admixture. Shea butter needs to be added to the melted second admixture away from the heat due to its melting point being very specific or else it will change the consistency of the product once cooled—i.e., if shea butter is melted in direct heat, it will become grainy once cooled. The combination may be continuously stirred until the shea butter is blended and infused with the previous admixture.

30-40 drops (approximately 1 ML) lavender oil may be introduced to the combination when the combination is at a temperature of approximately 100-155 degrees Fahrenheit. Though one embodiment includes adding the lavender oil at approximately 155 degrees Fahrenheit. The combination may be continuously stirred until the lavender oil is blended and infused with the third admixture, thereby forming the fourth admixture.

30-40 drops (approximately 1 ML) geranium oil may be introduced to the combination when the combination is at a temperature of approximately 100-155 degrees Fahrenheit, initiating the formation of a fifth admixture. The combination may be continuously stirred until the geranium oil is blended and infused with the fourth admixture, thereby forming the fifth admixture.

60-80 drops (approximately 2 ML) blue tansy oil may be introduced to the fifth admixture when at a temperature of approximately 100-155 degrees

Fahrenheit. The combination may be continuously stirred until the blue tansy oil is blended and infused, thereby forming a sixth admixture composition 10.

The sixth admixture composition 10 may then be urged into one or more containers 12. The container may be made of various plasticized material, such as but not limited to BPA plastic. The container 12 may be 4 oz container for the one embodiment disclosed herein. It being understood different embodiments may be formed through different ratios of weights/mass of ingredients, temperatures, and duration of the temperature, though the embodiment laid out herein is a preferred embodiment that benefits skin tissue. Optional replacements and swap outs of sourced items containers vat holdings, pots, pans, boilers, essential oils, butters, wax, that are sourced could be replaced or added with more quantity to make a greater yield of production while maintaining the same quality and upholding the same recipe, consistency, etc.

After adding the essential oils and mixing all of it together with a handheld mixer, the manufacturer can achieve a whipped consistency of the composition. An additional 2-3 drops of the blue tansy oil may be added to each container 12 to bring out a vibrancy of the present invention.

The composition formed by the above method steps may be applied dermally such as with a bandage, coverage, or various applicators. The present invention may be utilized during a tattoo/permanent makeup/micropigmentation process while working on an individual to promote a safe infusion of products during the healing process throughout and after the process is complete. It may also be applied for various treatments such as diaper rash, cut, eczema, sensitivity on the skin due to harsh climates, massage during a facial, to moisturize thirsty dry skin.

The component variation and steps are unique in execution and application so to render a desired, intended and specially purposed composition 10 built from beginning to end via the creation steps and protocols. For instance, the prepping and cleaning all the bottles and containers 12 in Step 1 lowers the number of potential contaminants that can affect the overall outcome. The progressive application of heat involved in Step 2 promotes melting while avoiding burning. In Step 3, the beeswax is introduced first as it is the densest in consistency e.g., thickest in the formulation and the process requires a breakdown of thickest to thinnest for the desired unique outcome. In step 4, the mango butter is not introduced into the same pot as the melted beeswax to combine and meltdown together. In Step 5 the extra virgin coconut oil is introduced into the beeswax and mango butter double boiling meltdown process). In Step 6, the shea butter is processed and melted down away from heat by removing the previous butters from heat (e.g., remove from the heating element) before introducing the shea butter at 165 degrees Fahrenheit and continuously stir in to blend with the other melted oils until the oils begin to infuse. In Step 7, checking the temperature is key to introducing the essential oil/s—e.g., the first introduction of the lavender essential oil is introduced at 155 degrees Fahrenheit. In Step 8, the geranium essential oil is added when the fourth admixture temperature is still held at 155 degrees Fahrenheit. In Step 9, the blue tansy essential oil and fifth admixture is still held at 155 degrees Fahrenheit. In Step 10, preferrable the manufacturer would stir and then pour. In Step 11, the container 12, after topping off the bottles an additional couple drops are added of the blue tansy to bring out the vibrancy of the invention.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the invention as set forth in the following claims.

What is claimed is:

1. A method for treating skin tissue, the method comprising dermally administering to a human or animal a portion of a composition comprising compositional components:
about 10.0 g of a beeswax;
about 18.75 g of a mango butter;
about 61.50 g of an extract of extra virgin coconut oil;
about 8.75 g of a shea butter;
about 0.25 g of lavender oil;
about 0.25 g of geranium oil; and
about 0.5 g of blue tansy oil.

2. The method of claim 1, wherein the beeswax, mango butter, and the shea 15 butter are melted prior to being mixed with other said compositional components.

3. A method for treating skin tissue, the method comprising dermally administering to a human or animal a portion of a composition comprising compositional components:
beeswax;
mango butter;
an extract of extra virgin coconut oil;
a shea butter;
lavender oil;
geranium oil; and
blue tansy oil, wherein said component were mixed in a sequence based on a density of each compositional components.

4. The method of claim 3, wherein the sequence is defined by most to least dense compositional components.

5. The method of claim 4, wherein the beeswax, mango butter, and the shea butter are melted prior to being mixed with other said compositional components.
